SEQUENCE LISTING (15 sequences)

SEQ ID NO: 1 - DNA, 1104 bp, Artificial sequence
  Synthetic
atggaccgcatcatccaatccccgggcaagtacatccaaggcgccgatgtgatcaaccgcctgggcgagtacctgaagccgctggccgagcgttggctggtcgtgggcgataagttcgtgctgggcttcgcccagagcaccgtggaaaagtcgttcaaggatgcgggcctcgtcgtggagatcgcgccgttcggcggtgagtgctcgcagaacgaaatcgaccgcctgcgcggcatcgcggaaaccgcgcagtgcggcgcgatcctgggcatcggcggcggcaagacgctggacaccgccaaggccctggcccattttatgggcgtcccggtggccatcgcccccaccatcgccagcacggacgcgccgtgctcggccctgagcgtcatctacaccgacgaaggcgagttcgaccgctatctgctcctgccgaataaccccaacatggtgatcgtggacaccaagatcgtggcgggcgccccggcccgcctgctggccgccggcattggcgacgcgctcgcgacgtggttcgaggcccgcgcgtgctcgcggagcggcgccaccacgatggcgggcggcaagtgtacccaggccgccctggccctggccgagctgtgctacaataccctgctggaagagggcgaaaaggccatgctggcggccgaacagcacgtggtgaccccggccctggagcgcgtgatcgaggcgaacacctacctgtcgggcgtgggcttcgaatccggcggtctggcggcggcccacgcggtccacaacggcctgaccgcgatccccgacgcccatcactactaccacggtgaaaaggtcgccttcggcaccctgacgcagctggtgctggagaacgccccggtcgaagagatcgaaacggtggcggccctctcccatgcggtgggcctcccgatcaccctcgcccagctggacatcaaggaagatgtccccgccaagatgcggatcgtcgccgaggccgcgtgcgccgagggtgaaacgattcacaacatgcccggcggcgcgacgccggaccaggtgtatgccgcgctgctcgtggcggaccagtatggccagcgcttcctgcaagagtgggagtga

SEQ ID NO: 2 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 3 - DNA, 1104 bp, Artificial sequence; identical to SEQ ID NO: 1
  Synthetic

SEQ ID NO: 4 - DNA, 1149 bp, Artificial sequence
  Synthetic
atggccaaccgcatgatcctgaacgaaaccgcctggttcggccgcggcgccgtcggtgcgctgacggacgaagtgaagcggcggggctaccaaaaggccctgatcgtgaccgacaagacgctggtccagtgcggcgtggtggccaaggtcaccgacaagatggatgccgcgggtctggcgtgggccatctacgacggcgtcgtcccgaacccgaccatcaccgtggtgaaggaaggcctgggcgtgttccagaacagcggcgccgactacctgatcgccatcggcggtggctccccgcaggacacgtgcaaggccatcggcatcatctcgaacaacccggagtttgccgacgtgcgctcgctggaaggcctgagccccacgaacaagccgtccgtgcccatcctcgcgattccgaccacggccggcacggccgccgaggtcaccatcaattacgtgatcacggacgaagagaagcgccgcaagttcgtctgcgtggacccccatgacattccccaggtcgcgttcattgacgcggacatgatggacggcatgccgccggcgctgaaggccgccaccggcgtcgatgccctcacccacgccatcgagggctatatcacccgcggcgcgtgggccctgacggatgccctgcacatcaaggccatcgagatcatcgccggtgccctgcgcggcagcgtggccggcgacaaagacgcgggcgaggaaatggccctgggccagtacgtcgcgggcatgggcttctcgaacgtgggcctcggcctggtgcatggcatggcgcacccgctgggcgcgttctacaacaccccgcacggtgtggcgaacgcgatcctgctgccccacgtgatgcgctacaacgccgacttcaccggcgagaagtatcgcgacatcgcccgtgtgatgggcgtgaaggtcgagggcatgtcgctggaagaagcgcgcaatgccgcggtcgaagcggtgttcgcgctgaaccgcgacgtgggcatcccgccccatctccgtgacgtgggcgtgcggaaggaagatatcccggccctcgcgcaggccgcgctggacgatgtctgcaccggcggcaatccgcgcgaggcgaccctggaagatatcgtcgagctgtatcacaccgcctggtga

SEQ ID NO: 5 - protein, 382 aa, Escherichia coli
MANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 6 - DNA, 1029 bp, Artificial sequence
  Synthetic
atgagcgtgttcgtgtcgggtgccaacggcttcatcgcccaacacattgtcgatctgctgctgaaggaagattacaaggtcatcggcagcgcccggtcccaggaaaaggccgagaacctcacggaagcgttcggcaacaacccgaagttctcgatggaagtcgtccccgacatctcgaagctggatgccttcgaccacgtctttcagaagcatggcaaggacatcaagatcgtcctgcatacggccagcccgttctgcttcgacatcaccgactcggagcgggacctcctgatcccggcggtcaacggcgtcaagggcatcctgcactcgatcaagaagtacgcggccgacagcgtggagcgcgtggtgctcacctccagctatgccgcggtgttcgacatggcgaaggaaaatgacaagtcgctgacctttaatgaagagtcctggaaccccgccacctgggagtcgtgccagagcgacccggtgaacgcgtactgcggctccaagaagttcgccgaaaaggccgcgtgggagttcctggaagagaaccgcgactcggtgaagttcgagctgacggccgtgaatccggtgtacgtgtttggcccccagatgttcgacaaggacgtgaagaagcacctgaacacgtcgtgcgagctggtgaacagcctgatgcatctgtcgcccgaggacaagatcccggagctgttcggcggctacatcgatgtccgcgacgtggcgaaggcccacctggtggcgttccagaagcgtgaaaccatcggccagcgcctgatcgtcagcgaggcgcgcttcaccatgcaagacgtgctggacatcctcaacgaagatttcccggtgctgaagggcaatatcccggtcggcaagccgggctcgggtgcgacccacaacacgctcggcgccaccctggacaacaagaagtccaagaagctgctgggcttcaagttccgcaacctgaaggaaacgatcgacgacaccgccagccagattctgaagtttgagggccgcatctga

SEQ ID NO: 7 - protein, 342 aa, Saccharomyces cerevisiae
MSVFVSGANGFIAQHIVDLLLKEDYKVIGSARSQEKAENLTEAFGNNPKFSMEVVPDISKLDAFDHVFQKHGKDIKIVLHTASPFCFDITDSERDLLIPAVNGVKGILHSIKKYAADSVERVVLTSSYAAVFDMAKENDKSLTFNEESWNPATWESCQSDPVNAYCGSKKFAEKAAWEFLEENRDSVKFELTAVNPVYVFGPQMFDKDVKKHLNTSCELVNSLMHLSPEDKIPELFGGYIDVRDVAKAHLVAFQKRETIGQRLIVSEARFTMQDVLDILNEDFPVLKGNIPVGKPGSGATHNTLGATLDNKKSKKLLGFKFRNLKETIDDTASQILKFEGRI

SEQ ID NO: 8 - DNA, 477 bp, Artificial sequence
  Synthetic
atgcaccaccatcatcaccacgcgctcatcatgaactcgaagaagaagattgccctggtcgcgcatgacaaccggaagaaggccctgatctcgtggtgcgaggccaactccgaggtgctgtcgaaccactcgctgtgcggcaccggcacgaccgccaagctgatcaaggaagcgaccggcctggaagtgttcccgtataagtccggtccgatgggcggcgaccagcagatcggcgccgcgatcgtcaacgaagatatcgacttcatgatcttcttctgggacccgctgacggcccagccgcacgacccggatgtgaaggccctgctgcgcatcagcgtgctgtacgacatccccatcgcgatgaatgagagcaccgccgagttcctcattaagagccccatcatgaaggaacaacacgagcgccacatcatcgactactaccagaagatccgcaaagacaacttttgatgaactagt

SEQ ID NO: 9 - protein, 149 aa, Clostridium acetobutylicum
MALIMNSKKKIALVAHDNRKKALISWCEANSEVLSNHSLCGTGTTAKLIKEATGLEVFPYKSGPMGGDQQIGAAIVNEDIDFMIFFWDPLTAQPHDPDVKALLRISVLYDIPIAMNESTAEFLIKSPIMKEQHERHIIDYYQKIRKDNF

SEQ ID NO: 10 - protein, 155 aa, Artificial sequence
  Synthetic
MHHHHHHALIMNSKKKIALVAHDNRKKALISWCEANSEVLSNHSLCGTGTTAKLIKEATGLEVFPYKSGPMGGDQQIGAAIVNEDIDFMIFFWDPLTAQPHDPDVKALLRISVLYDIPIAMNESTAEFLIKSPIMKEQHERHIIDYYQKIRKDNF

SEQ ID NO: 11 - DNA, 426 bp, Artificial sequence
  Synthetic
atgcaccaccatcatcaccacacccggccccgcatcgccctgatcgcccacgaccacaagaaggacgacattgtggccttcgccacccgccaccgcgcgttcctgtcgcagtgcgagctgctggcgacgggcaccaccggcggtcggctgatcgacgaagtgggcctggaagtcacgcgcatgctgtccggcccgtggggcggcgatctccagatcggcgcccaactggccgtcggcctcgtccgcgcggtggtgttcctccgcgatccgatgaccccgcagccgcacgagcccgacatcaacgcgctggtccgcgcgtgcgacgtgcataatgtcccgtgcgccacgaacgtggccaccgcggagctgctgatcgccggcctggcgcgtgaagtggacgaagccgcggccagctgatgaactagt

SEQ ID NO: 12 - protein, 132 aa, Cupriavidus necator
MTRPRIALIAHDHKKDDIVAFATRHRAFLSQCELLATGTTGGRLIDEVGLEVTRMLSGPWGGDLQIGAQLAVGLVRAVVFLRDPMTPQPHEPDINALVRACDVHNVPCATNVATAELLIAGLAREVDEAAAS

SEQ ID NO: 13 - protein, 138 aa, Artificial sequence
  Synthetic
MHHHHHHTRPRIALIAHDHKKDDIVAFATRHRAFLSQCELLATGTTGGRLIDEVGLEVTRMLSGPWGGDLQIGAQLAVGLVRAVVFLRDPMTPQPHEPDINALVRACDVHNVPCATNVATAELLIAGLAREVDEAAAS

SEQ ID NO: 14 - DNA, 1659 bp, Artificial sequence
  Synthetic
atgtcgcagttcttcttcaatcagcgcacccacctggtgagcgacgtgatcgatggcgcgatcatcgcctcgccctggaacaatctcgcccgcctggagtccgacccggccatccgcatcgtggtccgccgcgacctgaacaagaacaacgtcgccgtgatctcgggcggcggcagcggccacgagccggcgcacgtgggctttatcggcaagggcatgctgaccgcggccgtgtgcggcgacgtgttcgcgtccccgagcgtggacgcggtgctgacggcgatccaggccgtcaccggcgaggccggctgcctgctcatcgtgaagaactacaccggcgaccgtctgaacttcggcctggccgcggaaaaggcccggcgcctgggctacaatgtggagatgctgatcgtcggcgatgacatctccctgccggacaacaagcatccgcgcggtatcgcgggcaccatcctggtgcataagatcgccggctacttcgcggaacgcggctataacctcgccacggtcctgcgcgaggcccagtatgccgcgtcgaacacctttagcctgggcgtggccctgtcctcctgccatctgccccaggaaaccgacgccgcgccgcgccaccacccgggccacgcggagctgggcatgggcatccacggcgagccgggcgcctcggtgattgacacgcagaacagcgcccaagtcgtgaacctgatggtggacaagctgctggccgcgctgcccgaaacgggccgcctggccgtgatgattaacaatctgggcggcgtcagcgtggcggagatggcgatcatcacccgcgagctggcgtcgagccccctgcactcgcggattgactggctgatcggtccggcctccctggtcaccgcgctggacatgaagggcttctcgctcacggccatcgtgctggaagagtcgatcgaaaaggccctgctgaccgaagtcgaaacgtcgaactggccgaccccggtgcccccgcgcgagatcacgtgcgtcgtgagctcgcacgcgagcgcgcgcgtcgagttccagccgtcggcgaatgccctggtggccggcatcgtcgagctcgtcaccgccaccctgagcgacctggaaacgcatctgaacgccctggacgccaaggtcggcgatggcgatacgggctccaccttcgccgccgccgcgcgtgagatcgcctccctgctgcaccggcagcagctgcccctcaacaacctggcgacgctgttcgccctgatcggcgaacgcctgaccgtggtgatgggcggttcctcgggcgtcctcatgtcgatcttcttcaccgccgcgggccagaagctggaacagggcgcgaacgtcgtcgaggcgctcaacaccggtctggcccagatgaagttctacggcggcgccgacgaaggcgaccgcaccatgatcgacgcgctgcagccggcgctgacctcgctgctggcccagccgaagaacctccaagcggccttcgatgcggcccaggccggtgccgagcgcacgtgcctgtcctcgaaggcgaacgccggccgggccagctacctcagctcggaaagcctcctgggcaacatggaccccggcgcgcaacgtctggcgatggtgttcaaggccctggccgaaagcgagctgggctga

SEQ ID NO: 15 - protein, 552 aa, Citrobacter freundii
MSQFFFNQRTHLVSDVIDGAIIASPWNNLARLESDPAIRIVVRRDLNKNNVAVISGGGSGHEPAHVGFIGKGMLTAAVCGDVFASPSVDAVLTAIQAVTGEAGCLLIVKNYTGDRLNFGLAAEKARRLGYNVEMLIVGDDISLPDNKHPRGIAGTILVHKIAGYFAERGYNLATVLREAQYAASNTFSLGVALSSCHLPQETDAAPRHHPGHAELGMGIHGEPGASVIDTQNSAQVVNLMVDKLLAALPETGRLAVMINNLGGVSVAEMAIITRELASSPLHSRIDWLIGPASLVTALDMKGFSLTAIVLEESIEKALLTEVETSNWPTPVPPREITCVVSSHASARVEFQPSANALVAGIVELVTATLSDLETHLNALDAKVGDGDTGSTFAAAAREIASLLHRQQLPLNNLATLFALIGERLTVVMGGSSGVLMSIFFTAAGQKLEQGANVVEALNTGLAQMKFYGGADEGDRTMIDALQPALTSLLAQPKNLQAAFDAAQAGAERTCLSSKANAGRASYLSSESLLGNMDPGAQRLAMVFKALAESELG

(19) United States
(12) Patent Application Publication
(10) Pub. No.: US 2019/0233848 A1
(43) Pub. Date: Aug. 1, 2019

(54) METHODS AND MATERIALS FOR THE BIOSYNTHESIS OF DIOL ALCOHOLS AND RELATED COMPOUNDS

(71) Applicant: INVISTA NORTH AMERICA S.A.R.L., Wilmington, DE (US)

(72) Inventors: Nadia Fatma KADI, Redcar (GB); Daniel BAWDON, Redcar (GB); Alexander Brett FOSTER, Redcar (GB)

(21) Appl. No.: 16/264,768

(22) Filed: Feb. 1, 2019

Related U.S. Application Data
(60) Provisional application No. 62/624,863, filed on Feb. 1, 2018.

(51) Int. Cl. C12P 7/18; C12N 15/74; C12N 9/04; C12N 9/88; C12N 9/12

(52) U.S. Cl. CPC C12P 7/18; C12N 15/74; C12N 9/0006; C12Y 101/01006; C12Y 207/01029; C12Y 101/01283; C12Y 402/03003; C12N 9/88; C12N 9/1205; C12Y 101/01077

(57) ABSTRACT

Methods and materials for the production of diol alcohols, such as 1,2-propanediol (1,2-PD) and derivatives and compounds related thereto. Also provided are products produced in accordance with these methods and materials.

This patent application claims the benefit of priority from U.S. Provisional Application Ser. No. 62/624,863 filed Feb. 1, 2018, the content of this is herein incorporated by reference in its entirety.

### FIELD

The present invention relates to biosynthetic methods and materials for the production of diol alcohols, such as 1,2-propanediol (1,2-PD) and derivatives and other compounds related thereto. The present invention also relates to products biosynthesized or otherwise encompassed by these methods and materials.

Replacement of traditional chemical production processes relying on, for example fossil fuels and/or potentially toxic chemicals, with environmentally friendly (e.g., green chemicals) and/or “cleantech” solutions is being considered, including work to identify building blocks suitable for use in the manufacturing of such chemicals. See, “Conservative evolution and industrial metabolism in Green Chemistry”, Green Chem., 2018, 20, 2171-2191.

1,2-Propanediol (1,2-PD) is an important chemical in the production of polyesters, resins and polyurethanes, food applications, cosmetics, and is a diluent in some pharmaceuticals.

1,2-PD can be biosynthesized from dihydroxyacetone phosphate, an intermediate in glycolysis, by 2-4 exogenous genes in engineered strains of E. coli (Jain et al. ACS Synthetic Biology 2015 4:746-756; Jain et al. Microbial cell factories 2011 10:97; WO2010012604 A1), yeast Saccharomyces cerevisiae (Joon-Young et al. Journal of Microbial Biotechnology 2011 11(8):846-853), cyanobacterium Synechococcus elongatus (Li et al. Microbial Cell Factories 2013 12:4) and Corynebacterium glutamicum (Siebert et al., 2015 Biotechnology for biofuels 2015 8:91) using a carbohydrate such as glucose or glycerol (Jiang et al. Microbial Cell Factories 2014 13:165; Walther et al. Biotechnology Advances 2016 34:984-996; Matsubara et al. Journal of Bioscience and Bioengineering 2016 122(4):421-426; Bennett et al. Applied Microbial Biotechnology 2001 55:1-9) as the sole carbon source. This pathway also leads to production of 1-propanol from 1,2-propanediol when genes encoding a diol dehydratase/vitB12-dependent and aldo-keto reductase are present in E. coli (Jain et al., ACS Synthetic Biology 2015 4:746-756) (See FIG. 1).

To channel the carbon flux toward the engineered 1,2-PD pathway in E. coli, Jain et al. (ACS Synthetic Biology 2015 4:746-756) carried out a step-by-step study including the selection of optimum enzymes, the selection of a minimal set of optimum enzymes, the selection of gene deletions for channeling of the carbon flux, the increase of the NADH availability and the anaerobic growth condition of the cultures.

Addition of an extra gene encoding a dihydroxyacetone kinase to the 1,2-PD pathway has also been disclosed to allow an organism to synthesize 1,2-PD directly using glycerol as a carbon source instead of carbohydrate-based synthesis via dihydroxyacetone phosphate (DHAP) and dihydroxyacetone (Sánchez-Moreno et al. International Journal of Molecular Sciences 2015 16:27835-27849; Matsubara et al. Journal of Bioscience and Bioengineering 2016 122(4):421-426; Lee et al. Metabolic engineering 2016 36:48-56) (See FIG. 1). This pathway has the advantage of avoiding diversion of the central metabolite from a carbohydrate and provides information about the efficiency of the pathway from DHAP to 1,2-PD.

In addition, the deletion of genes such as gloA and ldhA, involved in lactate metabolism in E. coli, resulted in strains with improved production of 1,2-propanediol under anaerobic conditions (Jain et al. ACS Synthetic Biology 2015 4:746-756). The main by-products observed in E. coli, when engineered with a 1,2-PD pathway, were lactate (highest), acetate, formate, ethanol and succinate (Jain et al. ACS Synthetic Biology 2015 4:746-756). No 1,3-PD was observed in E. coli cultures with the 1,2-PD engineered pathway (WO2010012604 A1). A similar pathway engineered in E. coli has been shown to increase the 1,2-PD production when the gene gloA was deleted and the organism was cultured with oxygen using glycerol as a carbon source (WO2010012604 A1).

Only the last step from hydroxyacetone to 1,2-PD has been reported in C. necator for the in vivo study of hydrogen-driven coupling reaction (Oda et al. Microbial Cell Factories 2013 12:2).

Biosynthetic materials and methods, including organisms having increased production of 1,2-PD, derivatives thereof and compounds related thereto are needed.

### SUMMARY OF THE INVENTION

An aspect of the present invention relates to a process for biosynthesis of 1,2-PD and/or derivatives and/or compounds related thereto. The process comprises obtaining an organism capable of producing 1,2-PD and derivatives and compounds related thereto, altering the organism, and producing more 1,2-PD and derivatives and compounds related thereto in the altered organism as compared to the unaltered organism. In one nonlimiting embodiment, the organism is C. necator or an organism with one or more properties similar thereto. In one nonlimiting embodiment, the organism is altered to express glycerol dehydrogenase, 1,2-propanediol oxidoreductase, methylglyoxal reductase, methylglyoxal synthase and/or dihydroxyacetone kinase.

In one nonlimiting embodiment, the organism is altered to express a glycerol dehydrogenase. In one nonlimiting embodiment, the glycerol dehydrogenase comprises E. coli GldA (SEQ ID NO:2) or a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to an amino acid sequence set forth in SEQ ID NO: 2 or a functional fragment thereof. In one nonlimiting embodiment, the glycerol dehydrogenase is encoded by a nucleic acid sequence comprising E. coli gldA (SEQ ID NO:1 or SEQ ID NO:3) or a nucleic acid sequence encoding a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NOs: 1 or 3 or a functional fragment thereof. In one nonlimiting embodiment, the glycerol dehydrogenase is GldA classified in EC 1.1.1.6.

In one nonlimiting embodiment, the organism is altered to express a 1,2-propanediol oxidoreductase. In one nonlimiting embodiment, the 1,2-propanediol oxidoreductase comprises E. coli FucO (SEQ ID NO:5) or a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to an amino acid sequence set forth in SEQ ID NO: 5 or a functional fragment thereof. In one nonlimiting embodiment, the 1,2-propanediol oxidoreductase is encoded by a nucleic acid sequence comprising E. coli fucO (SEQ ID NO:4) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 4 or a functional fragment thereof. In one nonlimiting embodiment, the 1,2-propanediol oxidoreductase is FucO classified in EC 1.1.1.77.

In one nonlimiting embodiment, the organism is altered to express a methylglyoxal reductase. In one nonlimiting embodiment, the methylglyoxal reductase comprises S. cerevisiae Gre2 (SEQ ID NO:7) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 7 or a functional fragment thereof. In one nonlimiting embodiment, the methylglyoxal reductase is encoded by a nucleic acid sequence comprising S. cerevisiae gre2 (SEQ ID NO:6) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:6 or a functional fragment thereof. In one nonlimiting embodiment, the methylglyoxal reductase is Gre2 classified in EC 1.1.1.283.

In one nonlimiting embodiment, the organism is altered to express a methylglyoxal synthase. In one nonlimiting embodiment, the methylglyoxal synthase comprises Clostridium acetobutylicum MgsA (SEQ ID NO:9 or 10) or C. necator MgsA (SEQ ID NO:12 or 13) or a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to an amino acid sequence set forth in SEQ ID NOs: 9, 10, 12 or 13 or a functional fragment thereof. In one nonlimiting embodiment, the methylglyoxal synthase is encoded by a nucleic acid sequence comprising Clostridium acetobutylicum mgsA (SEQ ID NO:8) or C. necator mgsA (SEQ ID NO:11) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:8 or 11 or a functional fragment thereof. In one nonlimiting embodiment, the methylglyoxal is MgsA classified in EC 4.2.3.3.

In one nonlimiting embodiment, the organism is altered to express a dihydroxyacetone kinase. In one nonlimiting embodiment, the dihydroxyacetone kinase comprises Citrobacter freundii DhaK (SEQ ID NO:15) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 15 or a functional fragment thereof. In one nonlimiting embodiment, the dihydroxyacetone kinase is encoded by a nucleic acid sequence comprising Citrobacter freundii dhaK (SEQ ID NO:14) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:14 or a functional fragment thereof. In one nonlimiting embodiment, the dihydroxyacetone kinase is DhaK classified in EC 2.7.1.29.

In one nonlimiting embodiment, the nucleic acid sequence is codon optimized for C. necator.

In one nonlimiting embodiment, the organism is altered to express two or more of the enzymes of glycerol dehydrogenase, 1,2-propanediol oxidoreductase, methylglyoxal reductase, methylglyoxal synthase and/or dihydroxyacetone kinase as disclosed herein.

In one nonlimiting embodiment, the organism is altered to express three or more of the enzymes of glycerol dehydrogenase, 1,2-propanediol oxidoreductase, methylglyoxal reductase, methylglyoxal synthase and/or dihydroxyacetone kinase as disclosed herein.

In one nonlimiting embodiment, the organism is altered to express four or more of the enzymes of glycerol dehydrogenase, 1,2-propanediol oxidoreductase, methylglyoxal reductase, methylglyoxal synthase and/or dihydroxyacetone kinase as disclosed herein.

In one nonlimiting embodiment, the organism is altered to express glycerol dehydrogenase, 1,2-propanediol oxidoreductase, methylglyoxal reductase, methylglyoxal synthase and dihydroxyacetone kinase as disclosed herein.

In one nonlimiting embodiment, the organism is further altered to channel the carbon flux toward the intermediates of the 1,2-PD pathway. In one nonlimiting embodiment, one or more genes encoding for D-lactate dehydratases and/or D-lactate dehydrogenases and/or lactoylglutathione lyases are eliminated.

In one nonlimiting embodiment, the organism is further modified to eliminate phaCAB, involved in PHBs production and/or H16-A0006-9 encoding endonucleases thereby improving transformation efficiency.

Another aspect of the present invention relates to an organism altered to produce more 1,2-PD and/or derivatives and compounds related thereto as compared to the unaltered organism. In one nonlimiting embodiment, the organism is C. necator or an organism with properties similar thereto. In one nonlimiting embodiment, the organism is altered to express a glycerol dehydrogenase, 1,2-propanediol oxidoreductase, methylglyoxal reductase, methylglyoxal synthase and/or dihydroxyacetone kinase as disclosed herein.

In one nonlimiting embodiment, the organism is altered with a nucleic acid sequence codon optimized for C. necator.

In one nonlimiting embodiment, the organism is further altered to channel the carbon flux toward the intermediates of the 1,2-PD pathway. In one nonlimiting embodiment, one or more genes encoding for D-lactate dehydratases and/or D-lactate dehydrogenases and/or lactoylglutathione lyase are eliminated.

In one nonlimiting embodiment, the organism is further modified to eliminate phaCAB, involved in PHBs production and/or H16-A0006-9 encoding endonucleases thereby improving transformation efficiency.

In one nonlimiting embodiment, the organism is altered to express, overexpress, not express or express less of one or more molecules depicted in FIG. 1. In one nonlimiting embodiment, the molecule(s) comprise a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence corresponding to a molecule(s) depicted in FIG. 1, or a functional fragment thereof.

Another aspect of the present invention relates to bio-derived, bio-based, or fermentation-derived products produced from any of the methods and/or altered organisms disclosed herein. Such products include compositions comprising at least one bio-derived, bio-based, or fermentation-derived compound or any combination thereof, as well as bio-derived, bio-based, or fermentation-derived polymers or resins comprising these bio-derived, bio-based, or fermentation-derived compositions or compounds; molded substances obtained by molding the bio-derived, bio-based, or fermentation-derived polymers or resins or the bio-derived, bio-based; bio-derived, bio-based, or fermentation-derived formulations comprising the bio-derived, bio-based, or fermentation-derived compositions or compounds, polymers or resins, or the bio-derived, bio-based, or fermentation-derived molded substances, or any combination thereof; and bio-derived, bio-based, or fermentation-derived semi-solids or non-semi-solid streams comprising the bio-derived, bio-based, or fermentation-derived compositions or compounds, polymers or resins, molded substances or formulations, or any combination thereof.

Another aspect of the present invention relates to a bio-derived, bio-based or fermentation derived product biosynthesized in accordance with the exemplary central metabolism depicted in FIG. 1.

Yet another aspect of the present invention relates to exogenous genetic molecules of the altered organisms disclosed herein. In one nonlimiting embodiment, the exogenous genetic molecule comprises a codon optimized nucleic acid sequence encoding a glycerol dehydrogenase, 1,2-propanediol oxidoreductase, methylglyoxal reductase, methylglyoxal synthase and/or dihydroxyacetone kinase. In one nonlimiting embodiment, the nucleic acid sequence is codon optimized for C. necator. In one nonlimiting embodiment, the exogenous genetic molecule comprises a nucleic acid sequence encoding a glycerol dehydrogenase. In one nonlimiting embodiment the nucleic acid sequence comprises SEQ ID NO:1 or SEQ ID NO:3, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 3 or a functional fragment thereof, or a nucleic acid sequence encoding a polypeptide with similar enzymatic activities and exhibiting at least about 50%, 60%, 70%, 75%, 80%, 85%, 90%, 910, 92%, 93%, 94%, 95%, 96%, 97%, 98%, 99% or 99.5% sequence identity to the polypeptide encoded by the nucleic acid sequence set forth in SEQ ID NO: 1 or 3 or a functional fragment thereof. In one nonlimiting embodiment, the exogenous genetic molecule comprises a nucleic acid sequence encoding a 1,2-propanediol oxidoreductase. In one nonlimiting embodiment the nucleic acid sequence comprises SEQ ID NO:4, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:4 or a functional fragment thereof, or a nucleic acid sequence encoding a polypeptide with similar enzymatic activities and exhibiting at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in SEQ ID NO: 4 or a functional fragment thereof. In one nonlimiting embodiment, the exogenous genetic molecule comprises a nucleic acid sequence encoding a methylglyoxal reductase. In one nonlimiting embodiment the nucleic acid sequence comprises SEQ ID NO:6, a nucleic acid sequence exhibiting at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NO:6 or a functional fragment thereof, or a nucleic acid sequence encoding a polypeptide with similar enzymatic activities and exhibiting at least about 50%, 60%, 70%, 75%, 80%, 85%, 90%, 910, 92%, 93%, 94%, 950, 96%, 97%, 98%, 99% or 99.5% sequence identity to the polypeptide encoded by the nucleic acid sequence set forth in SEQ ID NO:6 or a functional fragment thereof. In one nonlimiting embodiment, the exogenous genetic molecule comprises a nucleic acid sequence encoding a methylglyoxal synthase. In one nonlimiting embodiment the nucleic acid sequence comprises SEQ ID NO:8 or 11, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:8 or 11 or a functional fragment thereof, or a nucleic acid sequence encoding a polypeptide with similar enzymatic activities and exhibiting at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in SEQ ID NO:8 or 11 or a functional fragment thereof. In one nonlimiting embodiment, the exogenous genetic molecule comprises a nucleic acid sequence encoding a dihydroxyacetone kinase. In one nonlimiting embodiment the nucleic acid sequence comprises SEQ ID NO:14, a nucleic acid sequence exhibiting at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NO:14 or a functional fragment thereof, or a nucleic acid sequence encoding a polypeptide with similar enzymatic activities and exhibiting at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in SEQ ID NO:14 or a functional fragment thereof. Additional nonlimiting examples of exogenous genetic molecules include expression constructs of, for example, a glycerol dehydrogenase, 1,2-propanediol oxidoreductase, methylglyoxal reductase, methylglyoxal synthase and/or dihydroxyacetone kinase and synthetic operons of, for example a glycerol dehydrogenase, 1,2-propanediol oxidoreductase, methylglyoxal reductase, methylglyoxal synthase and/or dihydroxyacetone kinase.

Yet another aspect of the present invention relates to means and processes for use of these means for biosynthesis of 1,2-PD and/or derivatives and/or compounds related thereto.

### BRIEF DESCRIPTION OF THE FIGURES

FIG. 1 is a schematic of the production of 1,2-PD in an organism with fructose as a carbon source.

FIGS. 2A and 2B shows nonlimiting examples of constructs having 3 (A) and 4 genes (B). The same canonical C. necator RBS sequence was introduced in front of each gene. The last gene in each construct had bases encoding for 6xHis in the N-terminal of the translated protein. (See Table 1 for genes codes).

FIG. 3 shows 1,2-PD production in the course of the cultures (Day 2 to 6) carried out in microreactor in the absence (left side of dashed line) and presence (right side of dashed line) of dhaK. Constructs are identified in Table 3 with number in parenthesis indicating the number of different biological replicates.

### DETAILED DESCRIPTION

The present invention provides processes for biosynthesis of 1,2-propanediol (1,2-PD), and/or derivatives thereof, and/or compounds related thereto, and organisms altered to increase biosynthesis of 1,2-PD, and/or derivatives thereof and/or compounds related thereto and organisms related thereto, exogenous genetic molecules of these altered organisms, and bio-derived, bio-based, or fermentation-derived products biosynthesized or otherwise produced by any of these methods and/or altered organisms.

In one aspect of the present invention, the carbon flux of the fructose metabolic node in an organism is redirected to produce 1,2-PD by alteration of the organism to express a glycerol dehydrogenase, 1,2-propanediol oxidoreductase, methylglyoxal reductase, methylglyoxal synthase and/or dihydroxyacetone kinase. Organisms produced in accordance with the present invention are useful in methods for biosynthesizing higher levels of 1,2-PD, derivatives thereof, and compounds related thereto.

For purposes of the present invention, by “1,2-propanediol (1,2-PD) and derivatives and compounds related thereto” it is meant to encompass propylene glycol, α-propylene glycol, 1,2-dihydroxypropane, methyl ethyl glycol and methylethylene glycol, as well as compounds derived from the same substrates and/or enzymatic reactions as 1,2-PD and having similar chemical structure as well as structural analogs wherein one or more functional groups of 1,2-PD are replaced with alternative substituents.

For purposes of the present invention, by “higher levels of 1,2-PD” it is meant that the altered organisms and methods of the present invention are capable of producing increased levels of 1,2-PD and derivatives and compounds related thereto as compared to the same organism without alteration. In one nonlimiting embodiment, levels are increased by 2-fold or higher.

For compounds containing carboxylic acid groups such as organic monoacids, hydroxyacids, amino acids and dicarboxylic acids, these compounds may be formed or converted to their ionic salt form when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate and/or bicarbonate, sodium hydroxide, ammonia and the like. The salt can be isolated as is from the system as the salt or converted to the free acid by reducing the pH to, for example, below the lowest pKa through addition of acid or treatment with an acidic ion exchange resin.

For compounds containing amine groups such as, but not limited to, organic amines, amino acids and diamine, these compounds may be formed or converted to their ionic salt form by addition of an acidic proton to the amine to form the ammonium salt, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid or muconic acid, and the like. The salt can be isolated as is from the system as a salt or converted to the free amine by raising the pH to, for example, above the highest pKa through addition of base or treatment with a basic ion exchange resin. Acceptable inorganic bases are known in the art and include aluminum hydroxide, calcium hydroxide, potassium hydroxide, sodium carbonate or bicarbonate, sodium hydroxide, and the like.

For compounds containing both amine groups and carboxylic acid groups such as, but not limited to, amino acids, these compounds may be formed or converted to their ionic salt form by either 1) acid addition salts, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include aluminum hydroxide, calcium hydroxide, potassium hydroxide, sodium carbonate and/or bicarbonate, sodium hydroxide, and the like, or 2) when an acidic proton present in the parent compound either is replaced by a metal ion, e.g., an alkali metal ion, an alkaline earth ion, or an aluminum ion; or coordinates with an organic base. Acceptable organic bases are known in the art and include ethanolamine, diethanolamine, triethanolamine, trimethylamine, N-methylglucamine, and the like. Acceptable inorganic bases are known in the art and include aluminum hydroxide, calcium hydroxide, potassium hydroxide, sodium carbonate, sodium hydroxide, ammonia and the like. The salt can be isolated as is from the system or converted to the free acid by reducing the pH to, for example, below the pKa through addition of acid or treatment with an acidic ion exchange resin. In one or more aspects of the invention, it is understood that the amino acid salt can be isolated as: i. at low pH, as the ammonium (salt)-free acid form; ii. at high pH, as the amine-carboxylic acid salt form; and/or iii. at neutral or midrange pH, as the free-amine acid form or zwitterion form.

In the process for biosynthesis of 1,2-PD and derivatives and compounds related thereto of the present invention, an organism capable of producing 1,2-PD and derivatives and compounds related thereto is obtained. The organism is then altered to produce more 1,2-PD and derivatives and compounds related thereto in the altered organism as compared to the unaltered organism.

In one nonlimiting embodiment, the organism is Cupriavidus necator (C. necator) or an organism with properties similar thereto. A nonlimiting embodiment of the organism is set for at lgcstandards-atcc with the extension .org/products/all/17699.aspx?geocountry=gb#generalinformation of the world wide web.

C. necator (previously called Hydrogenomonas eutrophus, Alcaligenes eutropha, Ralstonia eutropha, and Wautersia eutropha) is a Gram-negative, flagellated soil bacterium of the Betaproteobacteria class. This hydrogen-oxidizing bacterium is capable of growing at the interface of anaerobic and aerobic environments and easily adapts between heterotrophic and autotrophic lifestyles. Sources of energy for the bacterium include both organic compounds and hydrogen. Additional properties of C. necator include microaerophilicity, copper resistance (Makar, N. S. & Casida, L. E. Int. J. of Systematic Bacteriology 1987 37(4): 323-326), bacterial predation (Byrd et al. Can J Microbiol 1985 31:1157-1163; Sillman, C. E. & Casida, L. E. Can J Microbiol 1986 32:760-762; Zeph, L. E. & Casida, L. E. Applied and Environmental Microbiology 1986 52(4):819-823) and polyhydroxybutyrate (PHB) synthesis. In addition, the cells have been reported to be capable of both aerobic and nitrate dependent anaerobic growth. A nonlimiting example of a C. necator organism useful in the present invention is a C. necator of the H16 strain. In one nonlimiting embodiment, a C. necator host of the H16 strain with at least a portion of the phaCAB gene locus knocked out (ΔphaCAB) is used.

In another nonlimiting embodiment, the organism altered in the process of the present invention has one or more of the above-mentioned properties of Cupriavidus necator.

In another nonlimiting embodiment, the organism is selected from members of the genera Ralstonia, Wautersia, Cupriavidus, Alcaligenes, Burkholderia or Pandoraea.

For the process of the present invention, the organism is altered to express a glycerol dehydrogenase, 1,2-propanediol oxidoreductase, methylglyoxal reductase, methylglyoxal synthase and/or dihydroxyacetone kinase.

In one nonlimiting embodiment, the organism is altered to express a glycerol dehydrogenase. In one nonlimiting embodiment, the glycerol dehydrogenase comprises E. coli GldA (SEQ ID NO:2) or a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to an amino acid sequence set forth in SEQ ID NO: 2 or a functional fragment thereof. In one nonlimiting embodiment, the glycerol dehydrogenase is encoded by a nucleic acid sequence comprising E. coli gldA (SEQ ID NO:1 or SEQ ID NO:3) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NOs: 1 or 3 or a functional fragment thereof. In one nonlimiting embodiment, the glycerol dehydrogenase is GldA classified in EC 1.1.1.6.

In one nonlimiting embodiment, the organism is altered to express a 1,2-propanediol oxidoreductase. In one nonlimiting embodiment, the 1,2-propanediol oxidoreductase comprises E. coli FucO (SEQ ID NO:5) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 5 or a functional fragment thereof. In one nonlimiting embodiment, the 1,2-propanediol oxidoreductase is encoded by a nucleic acid sequence comprising E. coli fucO (SEQ ID NO:4) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 4 or a functional fragment thereof. In one nonlimiting embodiment, the 1,2-propanediol oxidoreductase is FucO classified in EC 1.1.1.77.

In one nonlimiting embodiment, the organism is altered to express a methylglyoxal reductase. In one nonlimiting embodiment, the methylglyoxal reductase comprises S. cerevisiae Gre2 (SEQ ID NO:7) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 7 or a functional fragment thereof. In one nonlimiting embodiment, the methylglyoxal reductase is encoded by a nucleic acid sequence comprising S. cerevisiae gre2 (SEQ ID NO:6) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:6 or a functional fragment thereof. In one nonlimiting embodiment, the methylglyoxal reductase is Gre2 classified in EC 1.1.1.283.

In one nonlimiting embodiment, the organism is altered to express a methylglyoxal synthase. In one nonlimiting embodiment, the methylglyoxal synthase comprises Clostridium acetobutylicum MgsA (SEQ ID NO:9 or 10) or C. necator MgsA (SEQ ID NO:12 or 13) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NOs: 9, 10, 12 or 13 or a functional fragment thereof. In one nonlimiting embodiment, the methylglyoxal synthase is encoded by a nucleic acid sequence comprising Clostridium acetobutylicum mgsA (SEQ ID NO:8) or C. necator mgsA (SEQ ID NO:11) or a nucleic acid sequence encoding a polypeptide with similar enzymatic activities exhibiting at least about 50%, 60%, 70%, 75%, 80%, 85%, 90%, 910, 92%, 93%, 94%, 95%, 960, 97%, 98%, 99% or 99.5% sequence identity to the nucleic acid sequence set forth in SEQ ID NO:8 or 11 or a functional fragment thereof. In one nonlimiting embodiment, the methylglyoxal synthase is MgsA classified in EC 4.2.3.3.

In one nonlimiting embodiment, the organism is altered to express a dihydroxyacetone kinase. In one nonlimiting embodiment, the dihydroxyacetone kinase comprises Citrobacter freundii DhaK (SEQ ID NO:15) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 15 or a functional fragment thereof. In one nonlimiting embodiment, the dihydroxyacetone kinase is encoded by a nucleic acid sequence comprising Citrobacter freundii dhaK (SEQ ID NO:14) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:14 or a functional fragment thereof. In one nonlimiting embodiment, the dihydroxyacetone kinase is DhaK classified in EC 2.7.1.29.

In one nonlimiting embodiment, the nucleic acid sequence is codon optimized for C. necator.

In one nonlimiting embodiment, the organism is altered to express two or more of the enzymes of glycerol dehydrogenase, 1,2-propanediol oxidoreductase, methylglyoxal reductase, methylglyoxal synthase and/or dihydroxyacetone kinase as disclosed herein.

In one nonlimiting embodiment, the organism is altered to express three or more of the enzymes of glycerol dehydrogenase, 1,2-propanediol oxidoreductase, methylglyoxal reductase, methylglyoxal synthase and/or dihydroxyacetone kinase as disclosed herein.

In one nonlimiting embodiment, the organism is altered to express four or more of the enzymes of glycerol dehydrogenase, 1,2-propanediol oxidoreductase, methylglyoxal reductase, methylglyoxal synthase and/or dihydroxyacetone kinase as disclosed herein.

In one nonlimiting embodiment, the organism is altered to express glycerol dehydrogenase, 1,2-propanediol oxidoreductase, methylglyoxal reductase, methylglyoxal synthase and dihydroxyacetone kinase as disclosed herein.

In one nonlimiting embodiment, the organism is further altered to channel the carbon flux toward the intermediates of the 1,2-PD pathway. In one nonlimiting embodiment, one or more genes encoding for D-lactate dehydratases such as, but not limited to, those classified in EC 4.2.1.130 and/or D-lactate dehydrogenases such as, but not limited to those classified in EC 1.1.1.28) and/or lactoylglutathione lyases such as, but not limited to, those classified in EC 4.4.1.5 are eliminated.

In one nonlimiting embodiment, the organism is further modified to eliminate phaCAB, involved in PHBs production and/or H16-A0006-9 encoding endonucleases thereby improving transformation efficiency as described in U.S. patent application Ser. No. 15/717,216, teachings of which are incorporated herein by reference.

In the process of the present invention, the altered organism is then subjected to conditions wherein 1,2-PD and derivatives and compounds related thereto are produced.

In one nonlimiting embodiment, micro-aerobic conditions are used.

In the process described herein, a fermentation strategy can be used that entails anaerobic, micro-aerobic or aerobic cultivation. A fermentation strategy can entail nutrient limitation such as nitrogen, phosphate or oxygen limitation.

Under conditions of nutrient limitation a phenomenon known as overflow metabolism (also known as energy spilling, uncoupling or spillage) occurs in many bacteria (Russell, 2007). In growth conditions in which there is a relative excess of carbon source and other nutrients (e.g. phosphorous, nitrogen and/or oxygen) are limiting cell growth, overflow metabolism results in the use of this excess energy (or carbon), not for biomass formation but for the excretion of metabolites, typically organic acids. In Cupriavidus necator a modified form of overflow metabolism occurs in which excess carbon is sunk intracellularly into the storage carbohydrate polyhydroxybutyrate (PHB). In strains of C. necator which are deficient in PHB synthesis this overflow metabolism can result in the production of extracellular overflow metabolites. The range of metabolites that have been detected in PHB deficient C. necator strains include acetate, acetone, butanoate, cis-aconitate, citrate, ethanol, fumarate, 3-hydroxybutanoate, propan-2-ol, malate, methanol, 2-methyl-propanoate, 2-methyl-butanoate, 3-methyl-butanoate, 2-oxoglutarate, meso-2,3-butanediol, acetoin, DL-2,3-butanediol, 2-methylpropan-1-ol, propan-1-ol, lactate 2-oxo-3-methylbutanoate, 2-oxo-3-methylpentanoate, propanoate, succinate, formic acid and pyruvate. The range of overflow metabolites produced in a particular fermentation can depend upon the limitation applied (e.g. nitrogen, phosphate, oxygen), the extent of the limitation, and the carbon source provided (Schlegel, H. G. & Vollbrecht, D. Journal of General Microbiology 1980 117:475-481; Steinbüchel, A. & Schlegel, H. G. Appl Microbiol Biotechnol 1989 31: 168; Vollbrecht et al. Eur J Appl Microbiol Biotechnol 1978 6:145-155; Vollbrecht et al. European J. Appl. Microbiol. Biotechnol. 1979 7: 267; Vollbrecht, D. & Schlegel, H. G. European J. Appl. Microbiol. Biotechnol. 1978 6: 157; Vollbrecht, D. & Schlegel, H. G. European J. Appl. Microbiol. Biotechnol. 1979 7: 259).

Applying a suitable nutrient limitation in defined fermentation conditions can thus result in an increase in the flux through a particular metabolic node. The application of this knowledge to C. necator strains genetically modified to produce desired chemical products via the same metabolic node can result in increased production of the desired product.

A cell retention strategy using a ceramic hollow fiber membrane can be employed to achieve and maintain a high cell density during fermentation. The principal carbon source fed to the fermentation can derive from a biological or non-biological feedstock. The biological feedstock can be, or can derive from, monosaccharides, disaccharides, lignocellulose, hemicellulose, cellulose, paper-pulp waste, black liquor, lignin, levulinic acid and formic acid, triglycerides, glycerol, fatty acids, agricultural waste, thin stillage, condensed distillers' solubles or municipal waste such as fruit peel/pulp. The non-biological feedstock can be, or can derive from, natural gas, syngas, CO₂/H₂, CO, H₂, O₂, methanol, ethanol, non-volatile residue (NVR) a caustic wash waste stream from cyclohexane oxidation processes or waste stream from a chemical industry such as, but not limited to a carbon black industry or a hydrogen-refining industry, or petrochemical industry, a nonlimiting example being a PTA-waste stream.

In one nonlimiting embodiment, at least one of the enzymatic conversions of the 1,2-PD production method comprises gas fermentation within the altered Cupriavidus necator host, or a member of the genera Ralstonia, Wautersia, Alcaligenes, Burkholderia and Pandoraea, and other organism having one or more of the above-mentioned properties of Cupriavidus necator. In this embodiment, the gas fermentation may comprise at least one of natural gas, syngas, CO₂/H₂, CO, H₂, O₂, methanol, ethanol, non-volatile residue, caustic wash from cyclohexane oxidation processes, or waste stream from a chemical industry such as, but not limited to a carbon black industry or a hydrogen-refining industry, or petrochemical industry. In one nonlimiting embodiment, the gas fermentation comprises CO₂/H₂.

The methods of the present invention may further comprise recovering produced 1,2-PD or derivatives or compounds related thereto. Once produced, any method can be used to isolate the 1,2-PD or derivatives or compounds related thereto.

The present invention also provides altered organisms capable of biosynthesizing increased amounts of 1,2-PD and derivatives and compounds related thereto as compared to the unaltered organism. In one nonlimiting embodiment, the altered organism of the present invention is a genetically engineered strain of Cupriavidus necator capable of producing 1,2-PD and derivatives and compounds related thereto. In another nonlimiting embodiment, the organism to be altered is selected from members of the genera Ralstonia, Wautersia, Alcaligenes, Cupriavidus, Burkholderia and Pandoraea, and other organisms having one or more of the above-mentioned properties of Cupriavidus necator. In one nonlimiting embodiment, the present invention relates to a substantially pure culture of the altered organism capable of producing 1,2-PD and derivatives and compounds related thereto via a glycerol dehydrogenase, 1,2-propanediol oxidoreductase, methylglyoxal reductase, methylglyoxal synthase and dihydroxyacetone kinase pathway.

As used herein, a “substantially pure culture” of an altered organism is a culture of that microorganism in which less than about 40% (i.e., less than about 35%; 30%; 25%; 20%; 15%; 10%; 5%; 2%; 1%; 0.5%; 0.25%; 0.1%; 0.01%; 0.001%; 0.0001%; or even less) of the total number of viable cells in the culture are viable cells other than the altered microorganism, e.g., bacterial, fungal (including yeast), mycoplasmal, or protozoan cells. The term “about” in this context means that the relevant percentage can be 15% of the specified percentage above or below the specified percentage. Thus, for example, about 20% can be 17% to 23%. Such a culture of altered microorganisms includes the cells and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

Altered organisms of the present invention may comprise at least one genome-integrated synthetic operon encoding an enzyme.

In one nonlimiting embodiment, the altered organism is produced by integration of a synthetic operon encoding a glycerol dehydrogenase, 1,2-propanediol oxidoreductase, methylglyoxal reductase, methylglyoxal synthase and/or dihydroxyacetone kinase.

In one nonlimiting embodiment, the glycerol dehydrogenase is from E. coli. In one nonlimiting embodiment, the glycerol dehydrogenase comprises E. coli GldA (SEQ ID NO:2) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 2 or a functional fragment thereof. In one nonlimiting embodiment, the glycerol dehydrogenase is encoded by a nucleic acid sequence comprising E. coli gldA (SEQ ID NO:1 or SEQ ID NO:3) or a nucleic acid sequence encoding a polypeptide with similar enzymatic activities exhibiting at least about 50%, 60%, 70%, 75%, 80%, 85%, 90%, 910, 92%, 93%, 94%, 95%, (CO, 97%, 98%, 99% or 99.5% sequence identity to the nucleic acid sequence set forth in SEQ ID NOs: 1 or 3 or a functional fragment thereof. In one nonlimiting embodiment, the glycerol dehydrogenase is GldA classified in EC 1.1.1.6.

In one nonlimiting embodiment, the 1,2-propanediol oxidoreductase is from E. coli. In one nonlimiting embodiment, the 1,2-propanediol oxidoreductase comprises E. coli FucO (SEQ ID NO:5) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 5 or a functional fragment thereof. In one nonlimiting embodiment, the 1,2-propanediol oxidoreductase is encoded by a nucleic acid sequence comprising E. coli fucO (SEQ ID NO:4) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 4 or a functional fragment thereof. In one nonlimiting embodiment, the 1,2-propanediol oxidoreductase is FucO classified in EC 1.1.1.77.

In one nonlimiting embodiment, the methylglyoxal reductase is from S. cerevisiae. In one nonlimiting embodiment, the methylglyoxal reductase comprises S. cerevisiae Gre2 (SEQ ID NO:7) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 7 or a functional fragment thereof. In one nonlimiting embodiment, the methylglyoxal reductase is encoded by a nucleic acid sequence comprising S. cerevisiae gre2 (SEQ ID NO:6) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:6 or a functional fragment thereof. In one nonlimiting embodiment, the methylglyoxal reductase is Gre2 classified in EC 1.1.1.283.

In one nonlimiting embodiment, the methylglyoxal synthase is from Clostridium acetobutylicum. In one nonlimiting embodiment, the methylglyoxal synthase comprises Clostridium acetobutylicum MgsA (SEQ ID NO:9 or 10) or C. necator MgsA (SEQ ID NO:12 or 13) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NOs: 9, 10, 12 or 13 or a functional fragment thereof. In one nonlimiting embodiment, the methylglyoxal synthase is encoded by a nucleic acid sequence comprising Clostridium acetobutylicum mgsA (SEQ ID NO:8) or C. necator mgsA (SEQ ID NO:11) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:8 or 11 or a functional fragment thereof. In one nonlimiting embodiment, the methylglyoxal synthase is MgsA classified in EC 4.2.3.3.

In one nonlimiting embodiment, the dihydroxyacetone kinase is from Citrobacter freundii. In one nonlimiting embodiment, the dihydroxyacetone kinase comprises Citrobacter freundii DhaK (SEQ ID NO:15) or a polypeptide with simiar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to an amino acid sequence set forth in SEQ ID NO: 15 or a functional fragment thereof. In one nonlimiting embodiment, the dihydroxyacetone kinase is encoded by a nucleic acid sequence comprising Citrobacter freundii dhaK (SEQ ID NO:14) or a nucleic acid sequence encoding a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NO:14 or a functional fragment thereof. In one nonlimiting embodiment, the dihydroxyacetone kinase is DhaK classified in EC 2.7.1.29.

In one nonlimiting embodiment, the nucleic acid sequence is codon optimized for C. necator.

In one nonlimiting embodiment, the organism is altered to express two or more of the enzymes of glycerol dehydrogenase, 1,2-propanediol oxidoreductase, methylglyoxal reductase, methylglyoxal synthase and/or dihydroxyacetone kinase as disclosed herein.

In one nonlimiting embodiment, the organism is altered to express three or more of the enzymes of glycerol dehydrogenase, 1,2-propanediol oxidoreductase, methylglyoxal reductase, methylglyoxal synthase and/or dihydroxyacetone kinase as disclosed herein.

In one nonlimiting embodiment, the organism is altered to express four or more of the enzymes of glycerol dehydrogenase, 1,2-propanediol oxidoreductase, methylglyoxal reductase, methylglyoxal synthase and/or dihydroxyacetone kinase as disclosed herein.

In one nonlimiting embodiment, the organism is altered to express glycerol dehydrogenase, 1,2-propanediol oxidoreductase, methylglyoxal reductase, methylglyoxal synthase and dihydroxyacetone kinase as disclosed herein. In one nonlimiting embodiment, the organism is further altered to channel the carbon flux toward the intermediates of the 1,2-PD pathway. In one nonlimiting embodiment, one or more genes encoding for D-lactate dehydratases such as, but not limited to, those classified in EC 4.2.1.130 and/or D-lactate dehydrogenases such as, but not limited to those classified in EC 1.1.1.28) and/or lactoylglutathione lyases such as, but not limited to, those classified in EC 4.4.1.5 are eliminated.

In one nonlimiting embodiment, the organism is further modified to eliminate phaCAB, involved in PHBs production and/or H16-A0006-9 encoding endonucleases thereby improving transformation efficiency.

The percent identity (and/or homology) between two amino acid sequences as disclosed herein can be determined as follows. First, the amino acid sequences are aligned using the BLAST 2 Sequences (B12seq) program from the stand-alone version of BLAST containing BLASTP version 2.0.14. This stand-alone version of BLAST can be obtained from the U.S. government's National Center for Biotechnology Information web site (www with the extension ncbi.nlm.nih.gov). Instructions explaining how to use the Bl2seq program can be found in the readme file accompanying BLASTZ. B12seq performs a comparison between two amino acid sequences using the BLASTP algorithm. To compare two amino acid sequences, the options of Bl2seq are set as follows: —i is set to a file containing the first amino acid sequence to be compared (e.g., C:\seq1.txt); —j is set to a file containing the second amino acid sequence to be compared (e.g., C:\seq2.txt); —p is set to blastp; —o is set to any desired file name (e.g., C:\output.txt); and all other options are left at their default setting. For example, the following command can be used to generate an output file containing a comparison between two amino acid sequences: C:\B12seq-i c:\seq1.txt-j c:\seq2.txt-p blastp-o c:\output.txt. If the two compared sequences share homology (identity), then the designated output file will present those regions of homology as aligned sequences. If the two compared sequences do not share homology (identity), then the designated output file will not present aligned sequences. Similar procedures can be followed for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 90.11, 90.12, 90.13, and 90.14 is rounded down to 90.1, while 90.15, 90.16, 90.17, 90.18, and 90.19 is rounded up to 90.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the polypeptides or nucleic acid sequences described herein can also be used in the methods and organisms disclosed herein. The term “functional fragment” as used herein refers to a peptide fragment of a polypeptide or a nucleic acid sequence fragment encoding a peptide fragment of a polypeptide that has at least about 25% (e.g., at least about 30%; 40%; 50%; 60%; 70%; 75%; 80%; 85%; 90%; 95%; 98%; 99%; 100%; or even greater than 100%) of the activity of the corresponding mature, full-length, polypeptide. The functional fragment can generally, but not always, be comprised of a continuous region of the polypeptide, wherein the region has functional activity. Functional fragments may range in length from about 10% up to 99% (inclusive of all percentages in between) of the original full-length sequence.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagluttanin (HA), glutathione-S-transferase (GST), or maltose binding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Endogenous genes of the organisms altered for use in the present invention also can be disrupted to prevent the formation of undesirable metabolites or prevent the loss of intermediates in the pathway through other enzymes acting on such intermediates. In one nonlimiting embodiment, the organism used in the present invention is further altered to channel the carbon flux toward the intermediates of the 1,2-PD pathway. In one nonlimiting embodiment, one or more genes encoding for D-lactate dehydratases such as, but not limited to, those classified in EC 4.2.1.130 and/or D-lactate dehydrogenases such as, but not limited to those classified in EC 1.1.1.28) and/or lactoylglutathione lyases such as, but not limited to, those classified in EC 4.4.1.5 are eliminated. In one nonlimiting embodiment, the organism is further modified to eliminate phaCAB, involved in PHBs production and/or H16-A0006-9 encoding endonucleases thereby improving transformation efficiency.

Thus, as described herein, altered organisms can include exogenous nucleic acids encoding a glycerol dehydrogenase, 1,2-propanediol oxidoreductase, methylglyoxal reductase, methylglyoxal synthase and/or dihydroxyacetone kinase, as described herein, as well as modifications to endogenous genes.

The term “exogenous” as used herein with reference to a nucleic acid (or a protein) and an organism refers to a nucleic acid that does not occur in (and cannot be obtained from) a cell of that particular type as it is found in nature or a protein encoded by such a nucleic acid. Thus, a non-naturally-occurring nucleic acid is considered to be exogenous to an organism or host once utilized by or in the organism or host. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

The present invention also provides exogenous genetic molecules of the nonnaturally occurring organisms disclosed herein such as, but not limited to, codon optimized nucleic acid sequences, expression constructs and/or synthetic operons.

In one nonlimiting embodiment, the exogenous genetic molecule comprises a codon optimized nucleic acid sequence encoding a glycerol dehydrogenase, 1,2-propanediol oxidoreductase, methylglyoxal reductase, methylglyoxal synthase and/or dihydroxyacetone kinase. In one nonlimiting embodiment, the nucleic acid sequence is codon optimized for C. necator.

In one nonlimiting embodiment, the exogenous genetic molecule comprises a nucleic acid sequence encoding a glycerol dehydrogenase. In one nonlimiting embodiment the nucleic acid sequence comprises SEQ ID NO:1 or SEQ ID NO:3, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 3 or a functional fragment thereof, or a nucleic acid sequence encoding a polypeptide with similar enzymatic activities and exhibiting at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in SEQ ID NO: 1 or 3 or a functional fragment thereof.

In one nonlimiting embodiment, the exogenous genetic molecule comprises a nucleic acid sequence encoding a 1,2-propanediol oxidoreductase. In one nonlimiting embodiment the nucleic acid sequence comprises SEQ ID NO:4, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:4 or a functional fragment thereof, or a nucleic acid sequence encoding a polypeptide with similar enzymatic activities and exhibiting at least about 50%, 60%, 70%, 75%, 80%, 85%, 90%, 910, 92%, 93%, 94%, 95%, 96%, 97%, 98%, 99% or 99.5% sequence identity to the polypeptide encoded by the nucleic acid sequence set forth in SEQ ID NO: 4 or a functional fragment thereof.

In one nonlimiting embodiment, the exogenous genetic molecule comprises a nucleic acid sequence encoding a methylglyoxal reductase. In one nonlimiting embodiment the nucleic acid sequence comprises SEQ ID NO:6, a nucleic acid sequence exhibiting at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NO:6 or a functional fragment thereof, or a nucleic acid sequence encoding a polypeptide with similar enzymatic activities and exhibiting at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in SEQ ID NO:6 or a functional fragment thereof.

In one nonlimiting embodiment, the exogenous genetic molecule comprises a nucleic acid sequence encoding a methylglyoxal synthase. In one nonlimiting embodiment the nucleic acid sequence comprises SEQ ID NO:8 or 11, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:8 or 11 or a functional fragment thereof, or a nucleic acid sequence encoding a polypeptide with similar enzymatic activities and exhibiting at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in SEQ ID NO:8 or 11 or a functional fragment thereof.

In one nonlimiting embodiment, the exogenous genetic molecule comprises a nucleic acid sequence encoding a dihydroxyacetone kinase. In one nonlimiting embodiment the nucleic acid sequence comprises SEQ ID NO:14, a nucleic acid sequence exhibiting at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NO:14 or a functional fragment thereof, or a nucleic acid sequence encoding a polypeptide with similar enzymatic activities and exhibiting at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in SEQ ID NO:14 or a functional fragment thereof.

Additional nonlimiting examples of exogenous genetic molecules include expression constructs of, for example, a glycerol dehydrogenase, 1,2-propanediol oxidoreductase, methylglyoxal reductase, methylglyoxal synthase and/or dihydroxyacetone kinase and synthetic operons of, for example, a glycerol dehydrogenase, 1,2-propanediol oxidoreductase, methylglyoxal reductase, methylglyoxal synthase and/or dihydroxyacetone kinase.

C. necator organisms produced in accordance with the present invention produced 1,2-propanediol from fructose at 30 degrees during 6 days under oxygen-limited conditions (micro-aerobic). All genetic constructs made of 3 and 4 genes from a combination of the 6 original genes produced 1,2-PD under these conditions but with varying efficiency. The quantity of 1,2-PD increased with time for most of the constructs expressed in C. necator to reach a maximum of 20 ppm of product after 6 days of cultures under micro-aerobic condition. Under this growth condition, the highest yield of 1,2-PD was obtained for the constructs made of 4 genes when the dhaK gene encoding for a dihydroxyacetone kinase was present.

Also provided by the present invention are 1,2-PD and derivatives and compounds related thereto bioderived from an altered organism according to any of methods described herein.

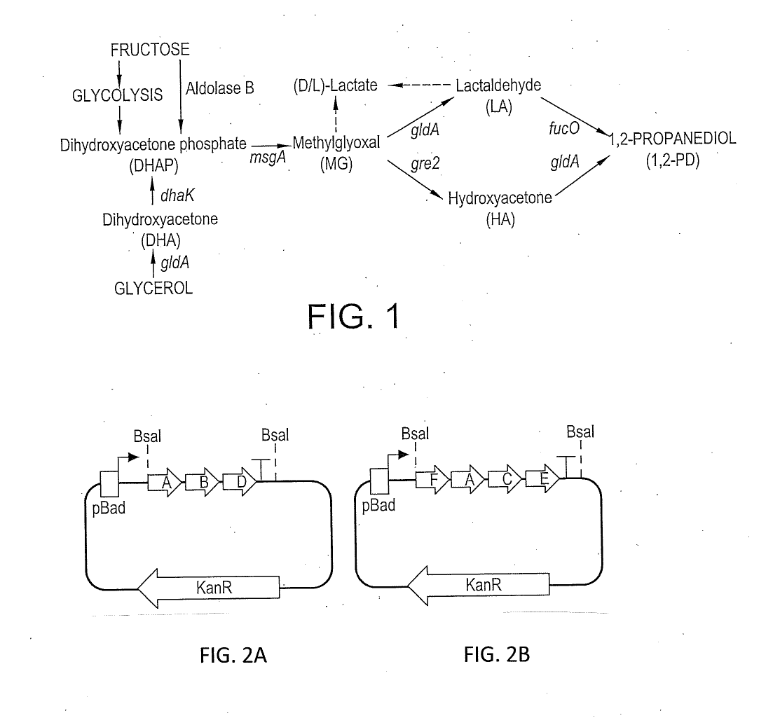

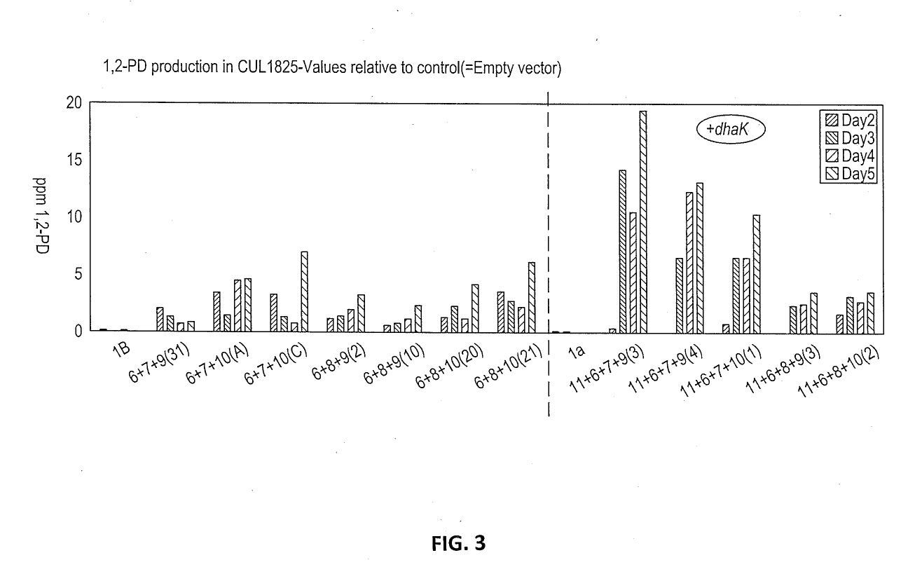

Further, the present invention relates to means and processes for use of these means for biosynthesis of 1,2-PD and/or derivatives and/or compounds related thereto. Nonlimiting examples of such means include altered organisms and exogenous genetic molecules as described herein as well as any of the molecules as depicted in FIG. 1.

In addition, the present invention provides bio-derived, bio-based, or fermentation-derived products produced using the methods and/or altered organisms disclosed herein. In one non-limiting embodiment, a bio-derived, bio-based or fermentation derived product is produced in accordance with the exemplary central metabolism depicted in FIG. 1. Examples of such products include, but are not limited to, compositions comprising at least one bio-derived, bio-based, or fermentation-derived compound or any combination thereof, as well as polymers or resins, molded substances, formulations and semi-solid or non-semi-solid streams comprising one or more of the bio-derived, bio-based, or fermentation-derived compounds or compositions, combinations or products thereof.

The following section provides further illustration of the methods and materials of the present invention. These Examples are illustrative only and are not intended to limit the scope of the invention in any way.

### Examples

### Gene Selection

The genes were first selected from biological resources and homology in C. necator. Genes gldA, fucO, mgsA are described by Jain et al. (ACS Synthetic Biology 2015 4:746-756 and Microbial cell factories 2011 10:97), Mampel et al. (WO2010012604 A1) and Siebert et al. (Biotechnology for biofuels, 2015 8:91). Gene Gre2 was described by Chen et al. (Yeast 2003 20:545-554). Gene dhaK was described by Sánchez et al. (International Journal of Molecular Sciences 2015 16:27835-27849) and Matsubara et al. (Journal of Bioscience and Bioengineering 2016 122(4): 421-426).

All the genes were codon-optimized for C. necator expression and the relevant features required for sub-cloning were added to the genes selected.

TABLE 1
Summary of the gene selection for 1,2-propanediol production in C. necator

| Code for | | | | |
|---|---|---|---|---|
| FIG. 2 | Uniprot | Enzyme name | EC | Organism |
| A | P0A9S5 | GldA, Glycerol dehydrogenase | 1.1.1.6 | Escherichia coli |
| | | | | (strain K12) |
| B | P0A9S1 | FucO, 1,2-propanediol | 1.1.1.77 | Escherichia coli |
| | | oxidoreductase | | (strain K12) |
| B | Q12068 | Gre2, Methylglyoxal reductase | 1.1.1.283 | Saccharomyces |
| | | | | cerevisiae |
| D | Q97IN6 | MgsA, Methylglyoxal synthase | 4.2.3.3 | Clostridium |
| | | | | acetobutylicum |
| | | | | (Ca) |
| E | Q0KD43 | MgsA, Methylglyoxal synthase | 4.2.3.3 | Cupriavidus |
| | | | | necator H16 (Cn) |
| F | P45510 | DhaK, Dihydroxyacetone | 2.7.1.29 | Citrobacter freundii |
| | | kinase | | |

TABLE 2
Summary of gene and protein features

| Enzyme name | Bps | AAs | MW (da) | pI |
|---|---|---|---|---|
| GldA, Glycerol dehydrogenase | 1104 | 367 | 38711.4 | 4.60 |
| FucO, 1,2-propanediol oxidoreductase | 1149 | 382 | 40512.6 | 4.92 |
| Gre2, Methylglyoxal reductase | 1020 | 342 | 38168.8 | 5.72 |
| MgsA, Methylglyoxal synthase (Ca) | 450 | 149 | 16805.1 | 6.22 |
| N-terminal His-tag version----------- | | 155 | 17628.0 | 6.65 |
| MgsA, Methylglyoxal synthase (Cn) | 399 | 132 | 14097.7 | 5.61 |
| N-terminal His-tag version----------- | | 138 | 14920.8 | 6.26 |
| DhaK, dihydroxyacetone kinase | 1659 | 552 | 57939.0 | 5.22 |

### Pathway Construction

The pBBR1-1B vector was used for producing constructs comprising 3 genes (FIG. 2A) and pBBR1-1A was used for producing constructs comprising 4 gene constructs (FIG. 2B).

Standard cloning techniques such as described, for example in Green and Sambrook, Molecular Cloning, A Laboratory Manual, Nov. 18, 2014 were used to assemble the 6 genes into 8 constructs (See Table 3).

TABLE 3
Gene combinations for 1,2-propanediol pathway
Gene name

gldA −> fucO −> msgA (Ca)
gldA −> gre2 −> msgA (Ca)
gldA −> fucO −> msgA (Cn)
gldA −> gre2 −> msgA (Cn)
dhaK −> gldA* −> fucO −> msgA (Ca)
dhaK −> gldA* −> gre2 −> msgA (Ca)
dhaK −> gldA* −> fucO −> msgA (Cn)
dhaK −> gldA* −> gre2 −> msgA (Cn)
*gldA required different features for sub-cloning with dhaK.

The 8 constructs were verified by PCR and sequencing. The PCR products were used as templates for sequencing to confirm the genes junctions of the constructs.

### C. Necator Deletion Strains

Three deletion strains were constructed (see Table 4):
1. ΔgloA1 (H16_A0517) (encoding for a lactoylglutathione lyase (EC 4.4.1.5));
2. ΔldhA1 ΔldhA2 (H16_A1681, H16_A1682)-(Encoding for D-lactate dehydrogenases (EC 1.1.1.28)); and
3. ΔldhA1 A1dhA2 ΔgloA1
   The base strain used to construct these strains was C. necator H16 ΔA0006-9 ΔphaCAB.

Genes ldhA1 and 1dhA2 are adjacent in the genome of C. necator. Therefore both genes were deleted using a single knockout plasmid. The gene 1dhA2 appears to be a direct gene duplication of ldhA1 as the DNA sequence of both genes is identical. C. necator deletion strain ΔldhA1 A1dhA2 was used as the base strain to construct deletion strain ΔldhA1 A1dhA2 ΔgloA1. Genotype screening was confirmed by PCR and agarose gel analysis as described, for example in Green and Sambrook, Molecular Cloning, A Laboratory Manual, Nov. 18, 2014.

For the constructs, a knockout cassette in a high-copy number ampicillin-based vector flanked by BbsI restriction enzyme cut sites (GAAGAC) and appropriate features was used for sub-cloning. The construct was passaged through E. coli NEB5α, purified and used for sub-cloning with a C. necator compatible suicide plasmid to generate a genomic integration knockout plasmid for each target locus.

TABLE 4
Summary C. necator strains used in this study

| Organism | Genotype | Type of clone | Gene deleted |
|---|---|---|---|
| C. necator | ΔphaCAB ΔA0006-9 | Chromosomal | N/A |
| H16 | (clone 1) | deletion | |
| C. necator | ΔA0006-9 ΔphaCAB | Chromosomal | H16_A0517 |
| H16 | ΔgloA1 | deletion | |
| C. necator | ΔA0006-9 ΔphaCAB | Chromosomal | H16_A1681 & |
| H16 | ΔldhA1A2 | deletion | H16_A1682 |
| C. necator | ΔA0006-9 | Chromosomal | H16_A1681, |
| H16 | ΔphaCAB ΔldhA1A2 | deletion | H16_A1682 & |
| | ΔgloA1 | | H16_A0517 |

### Growth Condition for Microreactors Ambr®15 Cultures

A—Media Recipes

Media adapted from Peoples and Sinskey (J Biol Chem 1989 264:15298-15303) was used.

B—Preparation of the Cultures for Microreactor (Ambr®15)

Seed train preparation: Cultures were first incubated overnight and then subcultured and further incubated for 16 hours. These were used as a direct inoculum for the fermentation fed batch cultures.

Cultures in microreactors: The Sartorius Ambr®15F platform (microreactors) was used to screen pathway strains in a fed batch mode of operation. This system allows control of multiple variables such as dissolved oxygen and pH. Typically, the process conditions were standardized and run following Sartorius's instructions.

C—Sample Preparation

All samples/cultures were spun down and the supernatants filtered through 0.2 μm filter.

Fifty microliters of filtered sample were mixed with 450 uL of acetonitrile (MS-grade). The mixture was centrifuged at 10,000 g for 20 min and 450 μL of the supernatant transferred in a 2 mL glass vial with screw-cap prior to be analyzed by GC-MS.

### GC-MS Analysis of 1,2-Propanediol

### Method Validation for 1,2-Propanediol (& 1,3-Propanediol):

Due to matrix effects of the supernatant/media, samples were diluted 10 times with ACN. The supernatant/media to prepare the calibration curve was also diluted 10 times with ACN.

A calibration curve was prepared.

TABLE 5
GC PARAMETERS TO ANALYZE for 1,2-propanediol & 1,3-propanediol:
GCMS CONDITIONS

| PARAMETER | VALUE | |
|---|---|---|
| Carrier Gas | Helium at constant flow (2.5 mL/min) | |
| Injector | Split ratio | Splitless |
| | Temperature | 250° C. |
| Detector | Source Temperature | 230° C. |
| | Quad Temperature | 150° C. |
| | Interface | 260° C. |
| | Gain | 1 |
| | Scan Range | m/z 30-500 o 30-200 |
| | Threshold | 150 |
| | Scan Speed (A/D samples) | 4 |
| | Mode | SCAN and SIM |
| Solvent delay * | 4.00 min | |
| Oven Temperature | Initial T: 80° C. × 2.0 min | |
| Oven Ramp | 10° C./min to 115° C. for 1.0 min | |
| | 120° C./min to 260° C. for 10 min | |

| | |
|---|---|
| Injection volume | 1.0 μL of liq injection |
| Gas saver | On after 2 min |
| Concentration range (μg/ml) | 15.63-250 PPM |
| GC Column | DB-624 122-1334 Agilent: 30 m × 250 μm × 1.4 μm |

* This parameter may vary according to the features of the column.

TABLE 6
HS-GCMS parameters using a Gerstel MPS Autosampler:

| Compound | Ions monitored in SIM Mode (m/z) |
|---|---|
| 1,2-propanediol | 45/61 |
| 1,3-propanediol | 31/43/57 |

### Sequence Information for Sequences in Sequence Listing

TABLE 7

| SEQ | |
|---|---|
| ID | |
| NO: | Sequence Description |
| 1 | Nucleic acid sequence of glycerol dehydrogenase, NAD+ |
| | dependent AAC76927.2 Escherichia coli str. K-12 |
| 2 | Amino acid sequence of GldA Glycerol dehydrogenase > |
| | sp\|P0A9S5\|GLDA_ECOLI Glycerol dehydrogenase |
| | OS = Escherichia coli (strain K12) GN = gldA PE = 1 SV = 1 |
| 3 | Nucleic acid sequence of gldA glycerol dehydrogenase, |
| | NAD+ dependent AAC76927.2 Escherichia coli str. K-12 |
| | C. necator optimized |
| 4 | Nucleic acid sequence of fucO 1,2-propanediol |
| | oxidoreductase AAC75841.2 Escherichia coli str. K-12 |
| | C. necator optimized |
| 5 | Amino acid sequence of FucO 1,2-propanediol |
| | oxidoreductase > sp\|P0A9S1\|FUCO_ECOLI Lactaldehyde |
| | reductase OS = Escherichia coli (strain K12) GN = fucO PE = 1 |
| | SV = 2; |
| 6 | Nucleic acid sequence of Gre2 NADPH-dependent |
| | methylglyoxal reductase GRE2 (putative) CAA88277.1 |
| | Saccharomyces cerevisiae C. necator optimized |
| 7 | Amino acid sequence of Gre2 Methylglyoxal reductase > |
| | sp\|Q12068\|GRE2_YEAST NADPH-dependent methylglyoxal |
| | reductase GRE2 OS = Saccharomyces cerevisiae (strain ATCC |
| | 204508/S288c) GN = GRE2 PE = 1 SV = 1 |
| 8 | Nucleic acid sequence of mgsA (C.a.) methylglyoxal |
| | synthase Q97IN6 Clostridium acetobutylicum C. necator |
| | optimized |
| 9 | Amino acid sequence of MgsA(Ca) Methylglyoxal synthase > |
| | sp\|Q97IN6\|MGSA_CLOAB Methylglyoxal synthase |
| | OS = Clostridium acetobutylicum (strain ATCC 824/DSM 792/ |
| | JCM 1419/LMG 5710/VKM B-1787) GN = mgsA PE = 3 SV = 2 |
| 10 | Amino acid sequence of MgsA(Ca) with 6xHis in N-terminal |
| 11 | Nucleic acid sequence of mgsA (C.n.) methylglyoxal |
| | synthase CAJ92078.1 Ralstonia eutropha H16 C. necator |
| | optimized |
| 12 | Amino acid sequence of Mgsa(Cn) Methylglyoxal synthase > |
| | tr\|Q0KD43\|Q0KD43_CUPNH Methylglyoxal synthase |
| | OS = Cupriavidus necator (strain ATCC 17699/H16/DSM |
| | 428/Stanier 337) GN = mgsA PE = 3 SV = 1; |
| 13 | Amino acid sequence of Protein MgsA(Cn) with 6xHis in N- |
| | terminal |
| 14 | Nucleic acid sequence of dhaK dihydroxyacetone kinase |
| | AAB48843.1 Citrobacter freundii C. necator optimized |
| 15 | Amino acid sequence of DhaK Dihydroxyacetone kinase > |
| | sp\|P45510\|DHAK_CITFR Dihydroxyacetone kinase |
| | OS = Citrobacter freundii GN = dhaK PE = 1 SV = 3 |

1: A process for biosynthesis of 1,2-propanediol (1,2-PD), and/or derivatives thereof and/or compounds related thereto, said process comprising:
obtaining an organism capable of producing 1,2-PD, derivatives thereof and/or compounds related thereto;
altering the organism; and
producing more 1,2-PD, derivatives thereof and/or compounds related thereto by the altered organism as compared to the unaltered organism.

2: The process of claim 1 wherein the organism is C. necator or an organism with properties similar thereto.

3: The process of claim 1 wherein the organism is altered to express one or more enzymes of glycerol dehydrogenase, 1,2-propanediol oxidoreductase, methylglyoxal reductase, methylglyoxal synthase and/or dihydroxyacetone kinase.

4-7. (canceled)

8: The process of claim 3 wherein the glycerol dehydrogenase and/or 1,2-propanediol oxidoreductase is from E. coli, the methylglyoxal reductase is from S. Cerevisiae, the methylglyoxal synthase is from Clostridium acetobutylicum or C. necator, and/or the dihydroxyacetone kinase is from Citrobacter freundii.

9: The process of claim 3 wherein the glycerol dehydrogenase comprises SEQ ID NO:2 or a polypeptide with similar enzymatic activities exhibiting at least about 50% sequence identity to an amino acid sequence set forth in SEQ ID NO: 2 or a functional fragment thereof or is encoded by a nucleic acid sequence comprising SEQ ID NO:1 or SEQ ID NO:3 or a nucleic acid sequence encoding a polypeptide with similar enzymatic activities exhibiting at least about 50% sequence identity to the nucleic acid sequence set forth in SEQ ID NOs: 1 or 3 or a functional fragment thereof.

10-11. (canceled)

12: The process of claim 3 wherein the 1,2-propanediol oxidoreductase comprises SEQ ID NO:5 or a polypeptide with similar enzymatic activities exhibiting at least about 50% sequence identity to an amino acid sequence set forth in SEQ ID NO: 5 or a functional fragment thereof or is encoded by a nucleic acid sequence comprising SEQ ID NO:4 or a nucleic acid sequence encoding a polypeptide with similar enzymatic activities exhibiting at least about 50% sequence identity to the nucleic acid sequence set forth in SEQ ID NO: 4 or a functional fragment thereof.

13-14. (canceled)

15: The process of claim 3 wherein the methylglyoxal reductase comprises SEQ ID NO:7 or a polypeptide with similar enzymatic activities exhibiting at least about 50% sequence identity to an amino acid sequence set forth in SEQ ID NO: 7 or a functional fragment thereof or is encoded by a nucleic acid sequence comprising SEQ ID NO:6 or a nucleic acid sequence encoding a polypeptide with similar enzymatic activities exhibiting at least about 50% sequence identity to the nucleic acid sequence set forth in SEQ ID NO:6 or a functional fragment thereof.

16-17. (canceled)

18: The process of claim 3 wherein the methylglyoxal synthase comprises SEQ ID NO:9, 10, 12 or 13 or a polypeptide with similar enzymatic activities exhibiting at least about 50% sequence identity to an amino acid sequence set forth in SEQ ID NOs: 9, 10, 12 or 13 or a functional fragment thereof or is encoded by a nucleic acid sequence comprising SEQ ID NO:8 or SEQ ID NO:11 or a nucleic acid sequence encoding a polypeptide with similar enzymatic activities exhibiting at least about 50% sequence identity to the nucleic acid sequence set forth in SEQ ID NO:8 or 11 or a functional fragment thereof.

19-20. (canceled)

21: The process of claim 3 wherein the dihydroxyacetone kinase comprises SEQ ID NO:15 or a polypeptide with similar enzymatic activities exhibiting at least about 50% sequence identity to an amino acid sequence set forth in SEQ ID NO: 15 or a functional fragment thereof or is encoded by a nucleic acid sequence comprising SEQ ID NO:14 or a nucleic acid sequence encoding a polypeptide with similar enzymatic activities exhibiting at least about 50% sequence identity to the nucleic acid sequence set forth in SEQ ID NO:14 or a functional fragment thereof.

22. (canceled)

23: The process of claim 1 wherein the organism is further altered to channel the carbon flux toward the intermediates of the 1,2-PD pathway.

24: The process of claim 1 wherein one or more genes encoding for D-lactate dehydratases and/or D-lactate dehydrogenases and/or lactoylglutathione lyases are eliminated.

25: The process of claim 1 wherein the organism is further altered to eliminate phaCAB, involved in PHBs production and/or H16-A0006-9 encoding endonucleases thereby improving transformation efficiency.

26. (canceled)

27: An altered organism capable of producing more 1,2-PD, derivatives thereof and/or compounds related thereto as compared to an unaltered organism.

28: The altered organism of claim 27 which is C. necator or an organism with properties similar thereto.

29: The altered organism of claim 27 which expresses one or more of a glycerol dehydrogenase, 1,2-propanediol oxidoreductase, methylglyoxal reductase, methylglyoxal synthase and/or dihydroxyacetone kinase.

30-33. (canceled)

34: The altered organism of claim 29 wherein the glycerol dehydrogenase and/or 1,2-propanediol oxidoreductase is from E. coli, the methylglyoxal reductase is from S. Cerevisiae, the methylglyoxal synthase is from Clostridium acetobutylicum or C. necator, and/or the dihydroxyacetone kinase is from Citrobacter freundii.

35: The altered organism of claim 29 wherein the glycerol dehydrogenase comprises SEQ ID NO:2 or a polypeptide with similar enzymatic activities exhibiting at least about 50% sequence identity to an amino acid sequence set forth in SEQ ID NO: 2 or a functional fragment thereof or is encoded by a nucleic acid sequence comprising SEQ ID NO:1 or SEQ ID NO:3 or a nucleic acid sequence encoding a polypeptide with similar enzymatic activities exhibiting at least about 50% sequence identity to the nucleic acid sequence set forth in SEQ ID NOs: 1 or 3 or a functional fragment thereof.

36-37. (canceled)

38: The altered organism of claim 29 wherein the 1,2-propanediol oxidoreductase comprises SEQ ID NO:5 or a polypeptide with similar enzymatic activities exhibiting at least about 50% sequence identity to an amino acid sequence set forth in SEQ ID NO: 5 or a functional fragment thereof or is encoded by a nucleic acid sequence comprising SEQ ID NO:4 or a nucleic acid sequence encoding a polypeptide with similar enzymatic activities exhibiting at least about 50% sequence identity to the nucleic acid sequence set forth in SEQ ID NO: 4 or a functional fragment thereof.

39-40. (canceled)

41: The altered organism of claim 29 wherein the methylglyoxal reductase comprises SEQ ID NO:7 or a polypeptide with similar enzymatic activities exhibiting at least about 50% sequence identity to an amino acid sequence set forth in SEQ ID NO: 7 or a functional fragment thereof or is encoded by a nucleic acid sequence comprising SEQ ID NO:6 or a nucleic acid sequence encoding a polypeptide with similar enzymatic activities exhibiting at least about 50% sequence identity to the nucleic acid sequence set forth in SEQ ID NO:6 or a functional fragment thereof.

42-43. (canceled)

44: The altered organism of claim 29 wherein the methylglyoxal synthase comprises SEQ ID NO:9, 10, 12 or 13 or a polypeptide with similar enzymatic activities exhibiting at least about 50% sequence identity to an amino acid sequence set forth in SEQ ID NOs: 9, 10, 11 or 12 or a functional fragment thereof or is encoded by a nucleic acid sequence comprising SEQ ID NO:8 or SEQ ID NO:11 or a nucleic acid sequence encoding a polypeptide with similar enzymatic activities exhibiting at least about 50% sequence identity to the nucleic acid sequence set forth in SEQ ID NO:8 or 11 or a functional fragment thereof.

45-46. (canceled)

47: The altered organism of claim 29 wherein the dihydroxyacetone kinase comprises SEQ ID NO:15 or a polypeptide with similar enzymatic activities exhibiting at least about 50% sequence identity to an amino acid sequence set forth in SEQ ID NO: 15 or a functional fragment thereof or is encoded by a nucleic acid sequence comprising SEQ ID NO:14 or a nucleic acid sequence encoding a polypeptide with similar enzymatic activities exhibiting at least about 50% sequence identity to the nucleic acid sequence set forth in SEQ ID NO:14 or a functional fragment thereof.

48. (canceled)

49: The altered organism of claim 27 wherein the organism is further altered to channel the carbon flux toward the intermediates of the 1,2-PD pathway.

50: The altered organism of claim 27 wherein one or more genes encoding for D-lactate dehydratases and/or D-lactate dehydrogenases and/or lactoylglutathione lyase are eliminated.

51: The altered organism of claim 27 wherein the organism is further altered to eliminate phaCAB, involved in PHBs production and/or H16-A0006-9 encoding endonucleases thereby improving transformation efficiency.

52. (canceled)

53: A bio-derived, bio-based, or fermentation-derived product produced from the method of claim 1, wherein said product comprises:
(i) a composition comprising at least one bio-derived, bio-based, or fermentation-derived compound or any combination thereof;
(ii) a bio-derived, bio-based, or fermentation-derived polymer or resin comprising the bio-derived, bio-based, or fermentation-derived composition or compound of (i), or any combination thereof;
(iii) a molded substance obtained by molding the bio-derived, bio-based, or fermentation-derived composition or compound of (i) or the bio-derived, bio-based, or fermentation-derived polymer or resin of (ii), or any combination thereof;
(iv) a bio-derived, bio-based, or fermentation-derived formulation comprising the bio-derived, bio-based, or fermentation-derived composition or compound of (i), the bio-derived, bio-based, or fermentation-derived polymer or resin of (ii), or the bio-derived, bio-based, or fermentation-derived molded substance of (iii), or any combination thereof; or
(v) a bio-derived, bio-based, or fermentation-derived semi-solid or a non-semi-solid stream, comprising the bio-derived, bio-based, or fermentation-derived composition or compound of (i), the bio-derived, bio-based, or fermentation-derived polymer or resin of (ii), the bio-derived, bio-based, or fermentation-derived formulation of (iii), or the bio-derived, bio-based, or fermentation-derived molded substance of (iv), or any combination thereof.

54: A bio-derived, bio-based or fermentation derived product produced in accordance with the central metabolism depicted in FIG. 1.

55: An exogenous genetic molecule of the altered organism of claim 27.

56: The exogenous genetic molecule of claim 55 comprising a codon optimized nucleic acid sequence or an expression construct or synthetic operon of a glycerol dehydrogenase, 1,2-propanediol oxidoreductase, methylglyoxal reductase, methylglyoxal synthase and/or dihydroxyacetone kinase.

57: The exogenous genetic molecule of claim 56 codon optimized for C. necator.

58: The exogenous genetic molecule of claim 55 comprising a nucleic acid sequence encoding a glycerol dehydrogenase, a 1,2-propanediol oxidoreductase, a methylglyoxal reductase, a methylglyoxal synthase or a dihydroxyacetone kinase.

59: The exogenous genetic molecule of claim 55 comprising SEQ ID NO:1 or SEQ ID NO:3, a nucleic acid sequence exhibiting at least about 50% sequence identity to the nucleic acid sequence set forth in SEQ ID NO: 1 or 3 or a functional fragment thereof, or a nucleic acid sequence encoding a polypeptide with similar enzymatic activities and exhibiting at least about 50% sequence identity to the polypeptide encoded by the nucleic acid sequence set forth in SEQ ID NO: 1 or 3 or a functional fragment thereof.

60. (canceled)

61: The exogenous genetic molecule of claim 55 comprising SEQ ID NO:4, a nucleic acid sequence exhibiting at least about 50% sequence identity to the nucleic acid sequence set forth in SEQ ID NO:4 or a functional fragment thereof, or a nucleic acid sequence encoding a polypeptide with similar enzymatic activities and exhibiting at least about 50% sequence identity to the polypeptide encoded by the nucleic acid sequence set forth in SEQ ID NO: 4 or a functional fragment thereof.

62. (canceled)

63: The exogenous genetic molecule of claim 55 comprising SEQ ID NO:6, a nucleic acid sequence exhibiting at least about 50% sequence identity to the nucleic acid sequence set forth in SEQ ID NO:6 or a functional fragment thereof, or a nucleic acid sequence encoding a polypeptide with similar enzymatic activities and exhibiting at least about 50% sequence identity to the polypeptide encoded by the nucleic acid sequence set forth in SEQ ID NO:6 or a functional fragment thereof.

64. (canceled)

65: The exogenous genetic molecule of claim 55 comprising SEQ ID NO:8 or 11, a nucleic acid sequence exhibiting at least about 50% sequence identity to the nucleic acid sequence set forth in SEQ ID NO:8 or 11 or a functional fragment thereof, or a nucleic acid sequence encoding a polypeptide with similar enzymatic activities and exhibiting at least about 50% sequence identity to the polypeptide encoded by the nucleic acid sequence set forth in SEQ ID NO:8 or 11 or a functional fragment thereof.

66. (canceled)

67: The exogenous genetic molecule of claim 55 comprising SEQ ID NO:14, a nucleic acid sequence exhibiting at least about 50% sequence identity to the nucleic acid sequence set forth in SEQ ID NO:14 or a functional fragment thereof, or a nucleic acid sequence encoding a polypeptide with similar enzymatic activities and exhibiting at least about 50% sequence identity to the polypeptide encoded by the nucleic acid sequence set forth in SEQ ID NO:14 or a functional fragment thereof.

68-69. (canceled)

70: A process for the biosynthesis of 1,2-propanediol (1,2-PD), derivatives thereof and/or compounds related thereto, said process comprising:
providing a means for producing 1,2-PD, derivatives thereof and/or compounds related thereto, and;
producing 1,2-PD, derivatives thereof and/or compounds related thereto with said means.

71: A process for biosynthesis of 1,2-PD, and derivatives thereof, and compounds related thereto, said process comprising:
a step for performing a function of altering an organism capable of producing 1,2-PD, derivatives thereof, and/or compounds related thereto such that the altered organism produces more 1,2-PD, derivatives thereof, and/or compounds compared to a corresponding unaltered organism; and
a step for performing a function of producing 1,2-PD, derivatives thereof, and/or compounds related thereto in the altered organism.

72-73. (canceled)
